(19) United States
(12) Patent Application Publication
(10) Pub. No.: US 2016/0228636 A1
(43) Pub. Date: Aug. 11, 2016

(54) FLUID ADMINISTRATION SYSTEM, VALVE, AND METHOD OF ADMINISTERING FLUID

(71) Applicants: Ana L. Maille, San Jose, CA (US); Joseph C. Maille, San Jose, CA (US)

(72) Inventors: Ana L. Maille, San Jose, CA (US); Joseph C. Maille, San Jose, CA (US)

(21) Appl. No.: 15/016,515

(22) Filed: Feb. 5, 2016

Related U.S. Application Data
(60) Provisional application No. 62/085,238, filed on Feb. 6, 2015.

(51) Int. Cl. A61M 5/168

(52) U.S. Cl. CPC A61M 5/16827; A61M 5/16813; A61M 2205/6081; A61M 2205/6018

(57) ABSTRACT

A fluid administration system includes a fluid pump, main tubing connected between a fluid storage container and an input of the fluid pump to supply main fluid to the fluid pump, and secondary tubing. A valve device is located along the secondary tubing. The valve device includes a fluid flow constriction device configured to allow fluid flow through the secondary tubing in an “open” position and to prevent fluid flow through the secondary tubing when in a “closed” position and a user-control device configured to control a position of the fluid flow constriction device between the “open” position and the “closed” position. The valve device includes a user feedback device configured to display an open and closed state of the fluid flow constriction device with a color code, having a first color representing the “open” position and a second color representing the “closed” position.

### CROSS REFERENCE TO RELATED APPLICATIONS

This application claims the benefit of an earlier filing date from U.S. Provisional Application Ser. No. 62/085,238 filed Feb. 6, 2015, the entire disclosure of which is incorporated herein by reference.

### BACKGROUND

In applications that use tubing to transmit fluids, valves and clamps are used to split or join fluid flows or to adjust fluid flows by reducing, increasing, or stopping fluid flow. When administering fluids through tubing, such as administering piggyback medications in an intravenous tube (IV), a roller clamp is used on the tubing which must be opened for the medications to be administered. Roller clamps may be small or it may be difficult to easily determine whether the roller clamp is open or closed. In particular, a primary or main IV tube is connected to an infusion pump, which controls the flow of IV fluids to a patient. Secondary tubing is connected to the main IV line, and does not have a variable flow control—instead, a clamp is used to prevent flow as the secondary fluids are being set up or changed, and to allow flow when the system is set up. However, since the clamp is small and difficult to see, it may be difficult to readily determine if a fluid is flowing from the secondary tubing, as desired. The infusion pump will continue pumping fluids from the main IV line, but if the clamp has not been opened, no fluid will flow from the secondary IV line.

Conventional clamps do not have any distinguishing features to allow a user to know if the clamp is open or closed. Thus, even though a staff person may enter a patient's room several times during a shift, the staff person may not notice that a clamp is closed, because the conventional roller clamp is small, typically about 1 centimeter long, and is made up of only one color, and therefore an open or closed state of the clamp is difficult to discern.

Some devices have tried to solve this problem in the field of IV tubing. For example, U.S. Pat. No. 8,672,875 describes a device that has an upstream pressure sensor and a processor for detecting if there is a fluid pressure increase when the secondary infusion is running. The processor is then supposed to generate an indicator or alarm if the fluid pressure does not increase. There are several problems with this device. It is not a simple solution, but includes installing processors and programming all current pumps to detect changes in pressure. There are hundreds of piggyback medications that have individual rates of infusion and therefore would correspond to different changes in pressure.

In addition, some piggyback medications run at a rate very similar to the main infusion so it would be hard for a sensor to detect a pressure change when the piggyback medication is running. Thus, there remains a need for a simple device that is easily manufactured to prevent errors when administering secondary fluids in tubing, such as piggyback medications on a main IV line.

### SUMMARY

Embodiments of the present invention include a fluid administration system. The fluid administration system may include a fluid pump, main tubing connected between a fluid storage container and an input of the fluid pump to supply main fluid to the fluid pump, and secondary tubing connected between a second fluid storage and the main tubing to supply a secondary fluid to the main fluid upstream of the fluid pump. A valve device is located along the secondary tubing. The valve device may include a fluid flow constriction device configured to allow fluid flow through the secondary tubing in an “open” position and to prevent fluid flow through the secondary tubing when in a “closed” position, a user-control device configured to control a position of the fluid flow constriction device between the “open” position and the “closed” position, and a user feedback device configured to display an open and closed state of the fluid flow constriction device with a color code, having a first color representing the “open” position and a second color representing the “closed” position.

In another embodiment of the invention, a valve device includes a fluid flow constriction device configured to allow fluid flow through secondary tubing in an “open” position and to prevent fluid flow through the secondary tubing when in a “closed” position. A user-control device is configured to control a position of the fluid flow constriction device between the “open” position and the “closed” position, and a user feedback device is configured to display an open and closed state of the fluid flow constriction device with a color code, having a first color representing the “open” position and a second color representing the “closed” position.

Another embodiment of the invention includes a method for applying a secondary fluid. The method includes initiating a flow of a primary fluid through primary tubing between a primary fluid container and a fluid pump and connecting secondary fluid tubing between a secondary fluid container and the primary tubing. The method further includes determining from a color indicator of a valve device connected along the secondary tubing whether a fluid flow through the secondary tubing is constricted. The method includes, based on determining from the color indicator of the valve device that the fluid flow is constricted, manipulating a user-control device of the valve device to change a state of the fluid flow from constricted to un-constricted flow through the secondary tubing.

### BRIEF DESCRIPTION OF THE DRAWINGS

Referring now to the drawings wherein like elements are numbered alike in the several Figures:

FIG. 1 illustrates a fluid administration system according to an embodiment of the invention;

FIG. 2A illustrates a block diagram of a valve feedback system according to one embodiment;

FIG. 2B illustrates a block diagram of a valve feedback system according to one embodiment;

FIG. 3A illustrates a valve feedback system according to one embodiment;

FIG. 3B illustrates a side cross-sectional view of the valve feedback system;

FIG. 3C illustrates a side cross-sectional view of the valve feedback system in a clamped state;

FIG. 4 illustrates a side cross-sectional view of a valve feedback system according to another embodiment;

FIG. 5A illustrates a valve feedback system according to an embodiment of the invention;

FIG. 5B illustrates a valve status indicator and user control according to an embodiment of the invention;

FIG. 6 illustrates a valve feedback system according to another embodiment of the invention;

FIG. 7 illustrates a valve feedback system according to another embodiment of the invention;

FIG. 8 illustrates a valve feedback system according to another embodiment of the invention;

FIG. 9 illustrates a valve feedback system according to another embodiment of the invention;

FIG. 10 illustrates a valve feedback system according to another embodiment of the invention;

FIG. 11 illustrates a valve feedback system according to another embodiment of the invention; and

FIG. 12 is a flowchart of a method for controlling a valve according to an embodiment of the invention.

### DETAILED DESCRIPTION

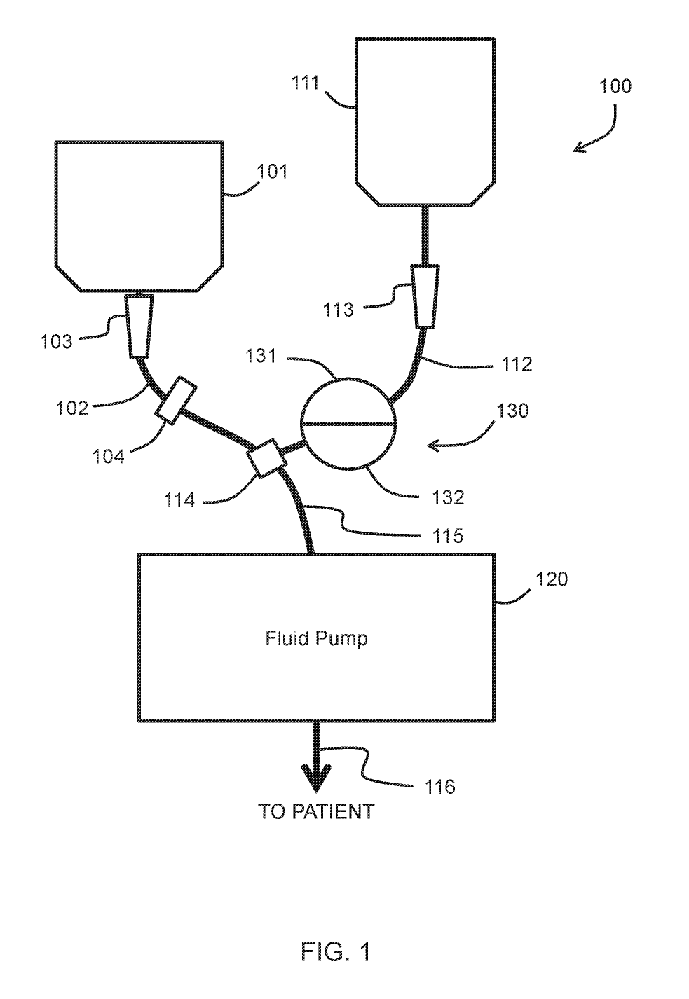

FIG. 1 illustrates a fluid administration system 100 according to an embodiment of the invention. The fluid administration system 100 includes a primary fluid container 101. A primary fluid tube 102 is connected to the primary fluid container 101 to receive fluid from the primary fluid container 101. A fluid pump 120 is connected to the primary fluid tube 102 at an opposite end from the primary fluid container 101 to receive fluid from the primary fluid tube 102.

In one embodiment in which the primary fluid container 101 contains medication or fluid for an intravenous infusion (IV), a primary fluid drip container 103 and primary fluid check valve 104 may be connected along the primary fluid tube 102.

The fluid administration system 100 includes a secondary fluid container 111. A secondary fluid tube 112 is connected to the secondary fluid container 111 and the primary fluid tube 102. In one embodiment, a joining device 114 connects the secondary fluid tube 112 to the primary fluid tube 111. In one embodiment, the joining device 114 is a passive joining device, such as a T-shaped or Y-shaped connector to allow fluid flow to continue through the primary fluid tube 102 while allowing fluid to flow from the secondary fluid tube 112 into the primary fluid tube 102. In other embodiments, the joining device 114 may include a valve mechanism.

The primary fluid from the primary fluid container 101 and the secondary fluid from the secondary fluid container 111 flow through the portion of the primary fluid tube 115 downstream from the joining device 114, such that the primary fluid and the secondary fluid flow from the joining device 114 to the fluid pump 120. The fluids flow from the fluid pump 120 to a destination via a fluid tube 116. For example, in one embodiment the fluid administration system 100 is an IV administration system, and the destination is a patient.

In an embodiment in which the fluid administration system 100 is an IV administration system, the secondary fluid contain 111 is elevated to be higher than the primary fluid container 101, and the fluid pump 120 is programmed to pump a predetermined amount of fluid, corresponding to the volume of the secondary fluid container 111, at a predetermined rate that may be different than a rate corresponding to the primary fluid from the primary fluid container 101. Because the secondary fluid container 111 is at an elevation higher than the primary fluid container 101, only the fluid from the secondary fluid container 111 flows through the joining device 114 into the portion 115 of tubing downstream of the joining device 114. When the pump 120 detects that the predetermined volume has been pumped, such that the secondary fluid container 111 is empty, the primary fluid begins flowing through the joining device 114 to the pump, and the pump 120 begins pumping at a rate corresponding to the primary fluid from the primary fluid container 101.

In one embodiment in which the secondary fluid container 111 contains medication or fluid for a piggyback infusion or medication, a secondary fluid drip container 113 may be connected along the secondary fluid tube 112.

In embodiments of the invention, a valve device 130 is connected along the secondary fluid tube 112. In one embodiment, the valve device 130 includes a first indicator region 131 and a second indicator region 132. The first indicator region 131 may be a first color, and the second indicator region 132 may be a second color, and the first and second colors may indicate “open” and “closed” states of a fluid flow valve in the valve device 130.

In one embodiment, the valve device 130 is an on/off valve device that does not have settings for varying a flow of fluids through the secondary fluid tube 112. In other words, in one embodiment, the valve device 130 has a user-control device having only two settings corresponding to “open” and “closed,” and the user-control device does not have any setting corresponding to any level of fluid flow between entirely open and entirely closed.

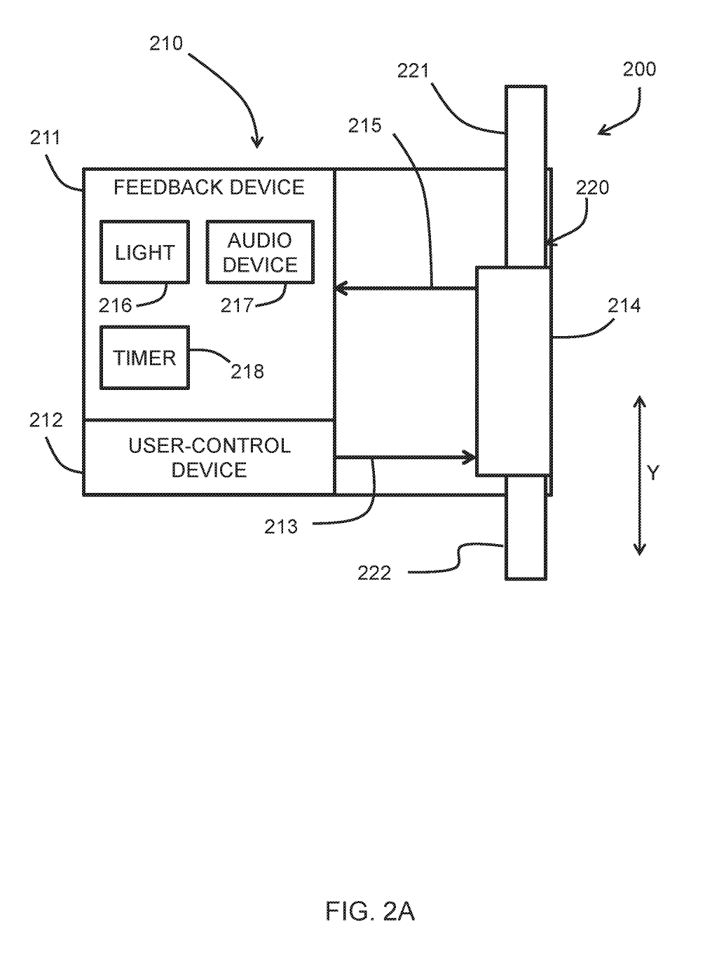

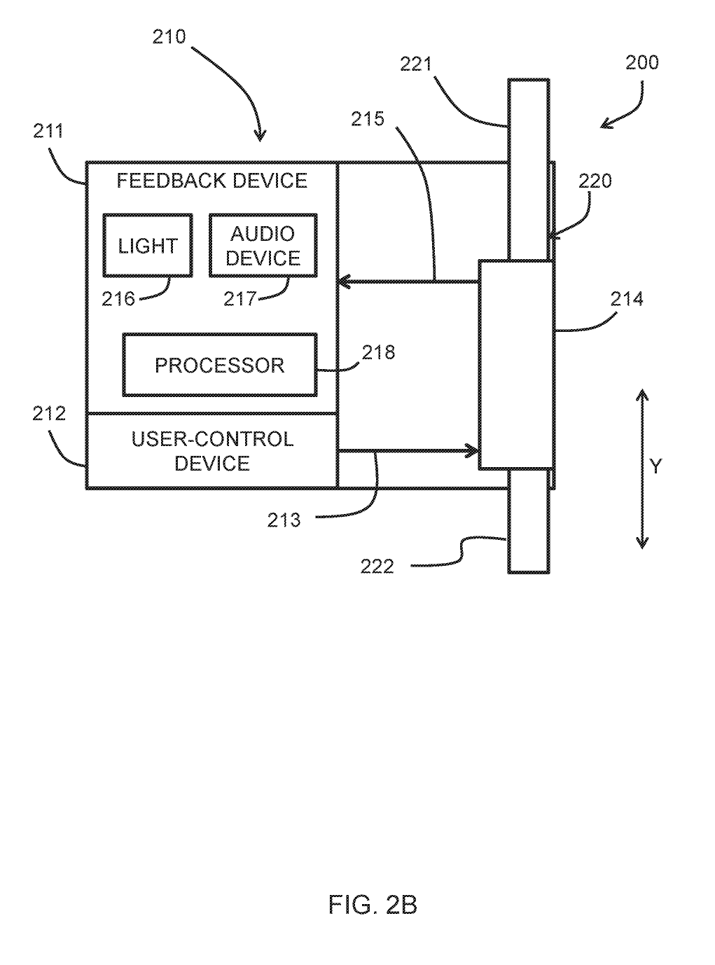

FIG. 2A is a block diagram of a valve feedback system 200 according to an embodiment of the invention. The system 200 includes a valve device 210 connected to a tube 220 having an input region 221 and an output region 222. In one embodiment, the valve device 210 is connected to the tube 220 without interrupting the continuity of the tube 220. For example, the valve device 210 may be attached around the tube 220 and may clamp the tube 220 to restrict flow through the tube 220, and may un-clamp the tube 220 to permit flow through the tube 220.

In another embodiment, the valve device 210 includes an input on the fluid flow constriction device 214 and an output on the fluid flow constriction device 214, and the tube 221 is a first tube connected to the input and the tube 222 is a second tube connected to the output.

The valve device 210 includes a feedback device 211 and a user-control device 212. A user manipulates the user-control device 212 to control an open or closed state of the fluid flow constriction device 214 via a valve control actuator 213, to permit or prevent flow of a fluid through the tube 220 in the fluid flow direction Y.

The valve device 210 includes a valve feedback actuator 215 connected to the feedback device 211 to control the feedback device 211 to indicate whether the fluid flow constriction device 214 is in an open state to permit fluid flow or a closed state to prevent fluid flow. In one embodiment, the feedback device 211 includes a first and second color corresponding to the open state and the closed state of the fluid flow constriction device 214. In one embodiment, the feedback device 211 includes an indicator highlighting or pointing to the first color to indicate that the fluid flow constriction device 214 is open and highlighting or pointing to the second color to indicate that the fluid flow constriction device 214 is closed.

In one embodiment, at least one of the first color and the second color are located physically on the user feedback device 211, such that manipulation of the user-control device 212 exposes the first color and conceals the second color based on the user-control device 212 being in a first position, and the user control device 212 exposes the second color and conceals the first color based on the user-control device 212 being in a second position. In another embodiment, controlling the user-control device 212 to open or close the fluid flow constriction device 214 highlights a first color or second color to indicate to the user the open or closed state, respectively.

In one embodiment, the first color corresponding to an open state of the fluid flow constriction device 214 is green. In one embodiment, the second color corresponding to a closed state of the fluid flow constriction device is one of red and black.

In one embodiment, the feedback device 211 includes a light 216 that illuminates to display at least one of the first and second colors based on whether the fluid flow constriction device 214 is open or closed.

In one embodiment, the feedback device 211 includes an audio device 217 to emit an alarm based on the user-control device 212 being in a position corresponding to the “closed” position of the fluid flow constriction device 214.

In one embodiment, the feedback device 211 includes a timer 218 that is set to detect an amount of time that the fluid flow constriction device is closed. If the timer 218 exceeds a predetermined time, one or both of the light 216 and the alarm from the audio device 217 may be triggered to provide notice to a user that the fluid-flow constriction device 214 is closed. For example, in an embodiment in which the valve device 210 is a valve on IV tubing in an IV administration system, the timer may give a user time to prepare or change a fluid container and to program a pump (such as the secondary fluid container 111 and fluid pump 120 of FIG. 1) without generating an alarm. However, once the predetermined time has been exceeded, corresponding to an estimated time necessary to prepare the IV tubing for IV administration, the alarm may be generated to ensure that the IV fluid or medication is provided to the patient.

According to various embodiments, the valve feedback actuator 215 may be one of a mechanical actuator or an electrical actuator. For example, in an embodiment in which the feedback device 211 includes one or both of the first and second colors painted, printed, or otherwise applied to or integrated in the structure of the feedback device 211, the valve feedback actuator 215 may be a mechanical mechanism. On the other hand, in an embodiment in which the feedback device 211 includes one or both of a light 216 and an audio device 217, the valve feedback actuator 215 may include a wire or electrical line.

In addition, it is understood that in an embodiment in which a feedback device 211 includes one or both of a light 216 and an audio device 217, the light 216 and audio device 217 may include conventional elements, such as wires, batteries, and any other electrical circuitry required to illuminate the light 216 or generate sound with the audio device 217.

In one embodiment, the valve device 210 is a simple mechanical or electrical device that does not include processing circuitry, such as a processor or other electronic processing unit. However, in an alternative embodiment shown in FIG. 2B, the feedback device 211 includes a processor 218 to control illumination of the light 216 or activation of the audio device 217. In addition, in such an embodiment, the valve feedback actuator 215 may include one or more sensors to detect a state of the fluid flow constriction device 214.

In one embodiment, the feedback actuator 215 includes a sensor to sense the presence of a fluid in the input region 221 of the tube, and the processor 218 controls the feedback device 211 to provide one or both of light and sound when the fluid is detected in the input region 221 and the fluid flow constriction device 214 is closed. In contrast, the processor 218 may control the feedback device 211 to not output one or both of light and sound when no fluid is detected in the input region 221. In other words, the processor 218 may control the feedback device 211 to output one or both of light and sound only when fluid is detected in the input region 221 of the tube 220 while the fluid flow constriction device 214 is closed.

According to embodiments of the invention, the user-control device 212 may be any device capable of controlling the fluid flow constriction device 214. According to one embodiment, the user-control device 212 includes a knob configured to be turned to a first position corresponding to the “open” position and to a second position corresponding to the “closed” position.

According to another embodiment, the user-control device 212 includes a user interface structure configured to be rotated around an axis perpendicular to a fluid-flow axis Y to change the fluid flow constriction device 214 between the “open” position and the “closed” position.

According to another embodiment, the user-control device 212 includes a rocker switch to change the fluid flow constriction device 214 between the “open” position and the “closed” position.

According to another embodiment, the user-control device 212 includes a user interface structure configured to rotate around a fluid-flow axis Y to change the fluid flow constriction device between the “open” position and the “closed” position.

According to another embodiment, the user-control device 212 includes a user interface structure configured to slide in a direction parallel to a fluid flow axis Y to change the fluid flow constriction device between the “open” position and the “closed” position.

In one embodiment, the feedback device 211 is integral with the user-control device 212. In other words, the user-control device 212 includes a feedback mechanism of the feedback device 212, such as printed, painted, or integral colors to show the status of the fluid flow constriction device 214, the light 216, or the audio device 217. In another embodiment, the feedback device 211 is separate from the user-control device 212. For example, the user-control device 212 may be a knob and the feedback device 211 may be a light 216 that is not a part of the knob.

While an embodiment has been described in which the feedback device 211 includes different colors to indicate an open or closed state of the fluid flow constriction device 214, in another embodiment, a prominent mechanical feature may indicate the state of the fluid flow constriction device 214 instead of color. For example, a tab or prong may extend prominently outward from the tube 220 when the fluid flow constriction device 214 is in the closed state, and may be co-linear with the flow axis Y, so as to not be prominently extending, when the fluid flow constriction device 214 is in the open state. In yet another embodiment, both colors and prominent mechanical features may be used to indicate an open or closed state of the fluid flow constriction device 214.

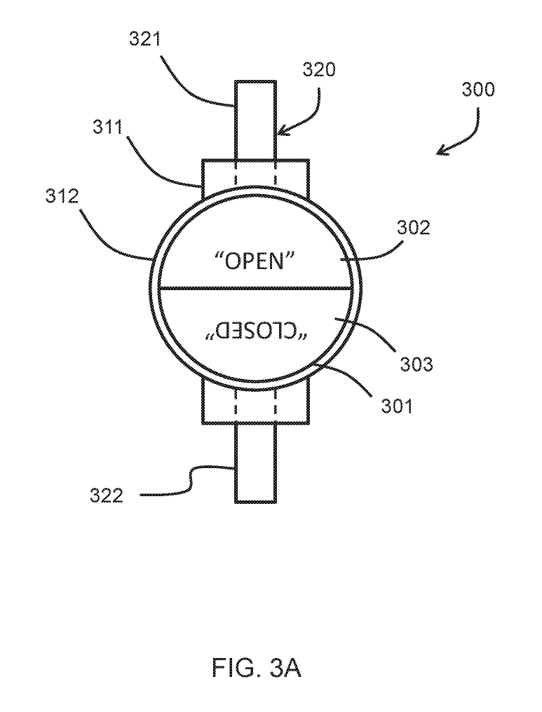

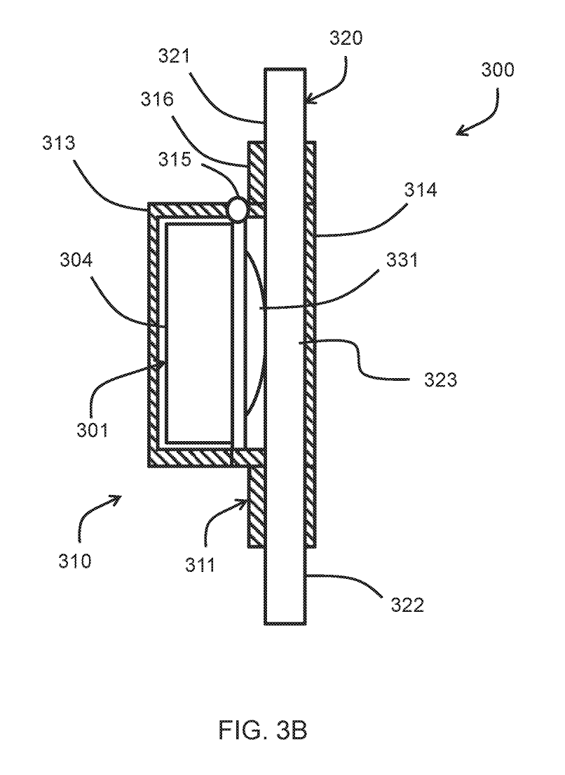

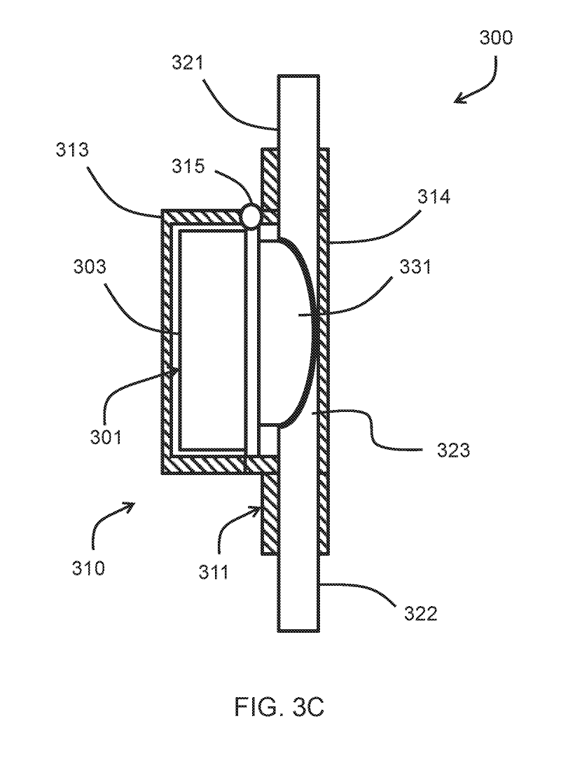

FIG. 3A illustrates a valve feedback system 300 according to one embodiment of the invention. The valve feedback system 300 includes a user-control device 301 including a first region 302 and a second region 303. In one embodiment, the first region 302 is a first color and the second region 303 is a second color. In one embodiment, the first region 302 is marked with the word “open” or another word used to signify that a fluid flow constriction device, such as the fluid flow constriction device 214 of FIG. 2A, is in an open state, and the second region 303 is marked with the word “closed” or another word to signify that the fluid flow constriction device is closed.

The valve feedback system 300 includes plastic casing 311 including a portion 312 of plastic casing surrounding the user-control device 301. The user control device 301 is attached to a tube 320 including an inflow portion 321 and outflow portion 322 and controls the flow of fluid through the tube 320.

In one embodiment, the user-control device 301 includes push-buttons, such that a user pushes the first region 302 to open a fluid flow constriction device and pushes the second region 302 to close the fluid flow constriction device. In another embodiment, the user-control device 301 is rotated relative to the casing 311 to control the fluid flow through the tube 320. For example, the inflow portion 321 of the tube may be oriented at a top position, such that when the first region 302 is on top of the second region 303, the fluid flow constriction device is open; and when the second region 303 is on top of the first region 302, the fluid flow constriction device is closed.

An example of such an embodiment in in a system for delivering medications of fluids via a secondary tubing line into a main IV tubing line. In such an embodiment, the inflow portion 321 is connected to a bag or container of fluid, such as medication, and so the inflow portion 321 must be oriented “up” to allow fluid to flow from the bag or container down to the fluid outflow portion 322 and into the main IV line.

In the embodiment illustrated in FIG. 3A, the user-control device 301 has an integrated feedback mechanism, such that the structure manipulated by a user to control fluid flow through the tube 320 also provides feedback to the user regarding the state of the fluid flow constriction device controlled by the user-control device 301.

FIG. 3B illustrates a side cross-sectional view of the valve feedback system 300 according to an embodiment in which a valve device 310 attaches to the tube 320 without breaking the tube 320, or in other words, without separating the inflow portion 321 from the outflow portion 322.

The valve feedback system 300 includes the user control device 301, and numeral 304 represents the front surface of the user control device 301. A hinge 315 may be used to open the plastic casing 311, to lift a cap 313 of the plastic casing from a base 316 of the plastic casing 311 to allow a user to access the user-control device 301. The cap 313 may be transparent to allow a user to see a condition or state of the fluid flow constriction device.

A portion 314 of the plastic casing 311 may cover the tube 320. In the embodiment illustrated in FIG. 3B, the fluid flow constriction device includes a clamp device 331 that is mechanically connected to the user control device 301 to control fluid flow through the tube 320. As illustrated in FIG. 3B, when the user-control device 301 is controlled to place the fluid flow constriction device in an open state, the clamp 331 does not press the tube 320 sufficient to constrict fluid flow through the tube 320.

However, as illustrated in FIG. 3C, when the user-control device 301 is controlled to place the fluid flow constriction device in a closed state, the clamp 331 presses the middle region 323 of the tube 320 sufficient to constrict fluid flow through the tube 320.

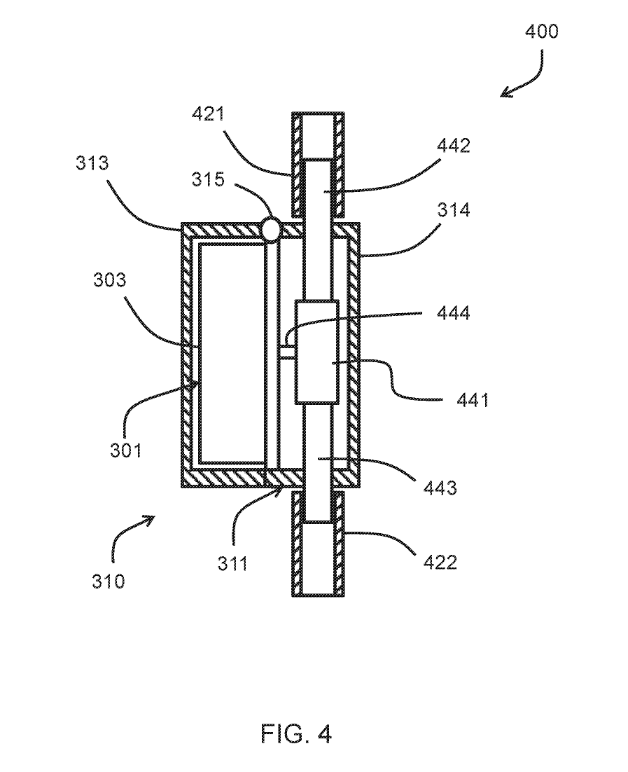

FIG. 4 illustrates an alternative embodiment, in which the valve device 310 includes an inlet portion 442 to connect to an inflow tube 421 and an outlet portion 443 to connect to an outflow tube 422. The inlet portion 442 is connected to a fluid flow constriction device 441, such as a valve, which is connected by an actuator 444 to the user-control device 301. Accordingly, the user-control device 301 controls the fluid flow constriction device 441 to either allow fluid to flow from the inlet 442 to the outlet 443, or to prevent fluid from flowing.

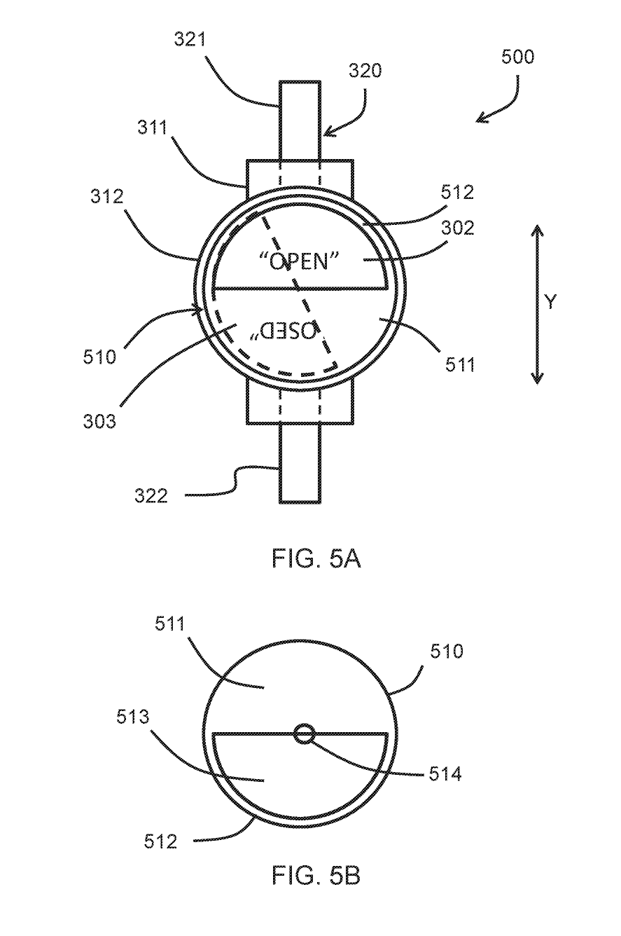

FIGS. 5A and 5B illustrate a valve feedback system 500 according to an embodiment of the invention. In the valve feedback system 500 illustrated in FIGS. 5A and 5B, the device used by a user to control fluid flow through a tube 320 is also used to provide feedback regarding the state of a fluid flow constriction device, such as the fluid flow constriction device 214 of FIG. 2A.

Referring to FIG. 5A, the valve feedback system 500 includes a valve status indicator 512, which is also a valve user control. The valve status indicator (and user control) 510 includes an obscuring portion 511, and an exposing portion 513 surrounded by an edge 512. When the first region 302 is located in the exposing region 513, as indicated by the solid lines in FIG. 5A, fluid is permitted to flow along the fluid flow axis Y through the tube 320. Accordingly, the second region 303 is obscured by the obscuring portion 511, providing feedback to the user that the fluid flow constriction device is “open.” In contrast, when the second region 303 is located in the exposing region 513, fluid is not permitted to flow along the fluid flow axis Y through the tube 320. Accordingly, the first region 302 is obscured by the obscuring portion 511, providing feedback to the user that the fluid flow constriction device is “closed.” The dashed lines of FIG. 5A show the valve status indicator 510 being transitioned between an “open” state and a “closed” state.

FIG. 5B illustrates the valve status indicator 510 without the rest of the valve feedback system 500, for purposes of clarity. As illustrated in FIG. 5B, the valve status indicator 510 may include an actuator 514 at a center of the valve status indicator 510, such that as the valve status indicator 510 is rotated, the actuator 514 is actuated to control a fluid flow constriction device. In alternative embodiments, an actuator may be connected to any portion of the valve status indicator 510, such as around an outside edge of the valve status indicator 510.

FIGS. 5A and 5B illustrate an embodiment in which fluid flow through a tube is controlled by rotating a control mechanism around an axis perpendicular to a fluid flow axis Y. However, embodiments of the invention encompass control mechanisms that control flow based on rotating control mechanisms around the fluid flow axis, based on sliding a control mechanism parallel to the fluid flow axis, based on controlling a switch or button, or based on any other control.

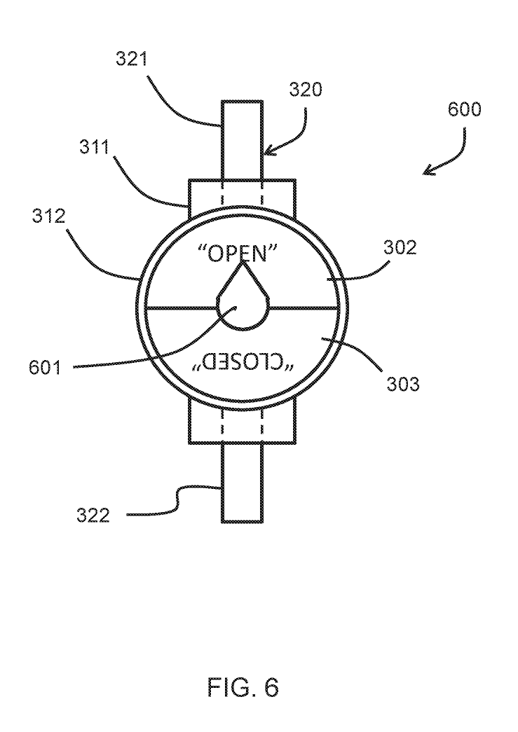

FIG. 6 illustrates a valve feedback system 600 according to another embodiment of the invention. The valve feedback system 600 of FIG. 6 is similar to the valve feedback system 300 illustrated in FIG. 3A, except in the embodiment illustrated in FIG. 6, a knob 601 points to the first region 302 or the second region 303 to indicate a status of a fluid flow constriction device. Accordingly, the knob 601 may both be controlled by a user to control flow, and may indicate the status of the fluid flow constriction device.

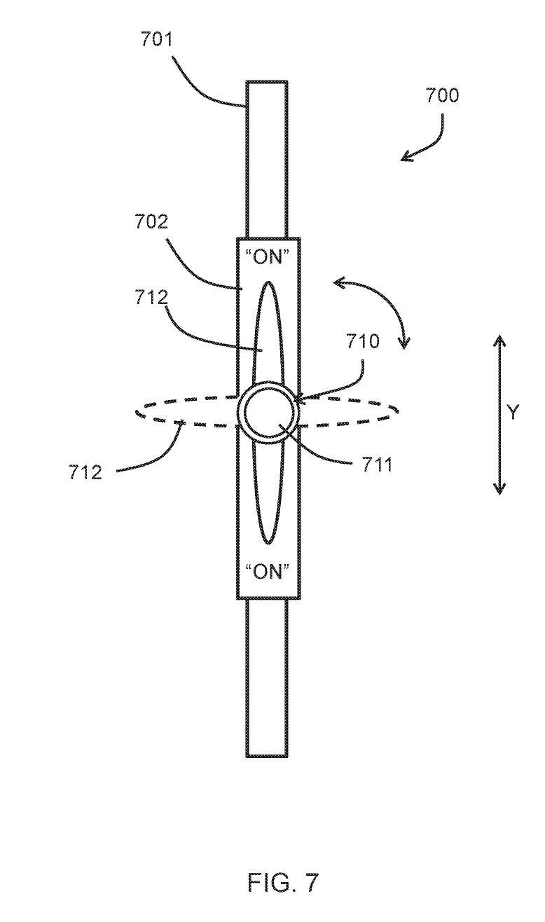

FIG. 7 illustrates a valve feedback system 700 according to another embodiment of the invention. The valve feedback system 700 includes tubing 701 and a valve device 702. The valve device 702 includes a knob 710, including a prong 712 which is manipulated by a user to control fluid flow through the tubing 701. The valve device 702 may be controlled such that when the prong 712 is aligned along the fluid flow axis Y, fluid is permitted to flow through the tubing 701, and when the prong is aligned perpendicularly to the fluid flow axis Y, fluid is not permitted to flow through the tubing 701. Accordingly, the position of the prong 712 relative to the fluid flow axis Y provides feedback to a user regarding whether fluid is flowing or permitted to flow through the valve device 702.

In addition, in one embodiment the valve device 702 further includes a valve status indicator 711, such as a light or speaker, to emit one or both of light and sound based on the position of the prong 712.

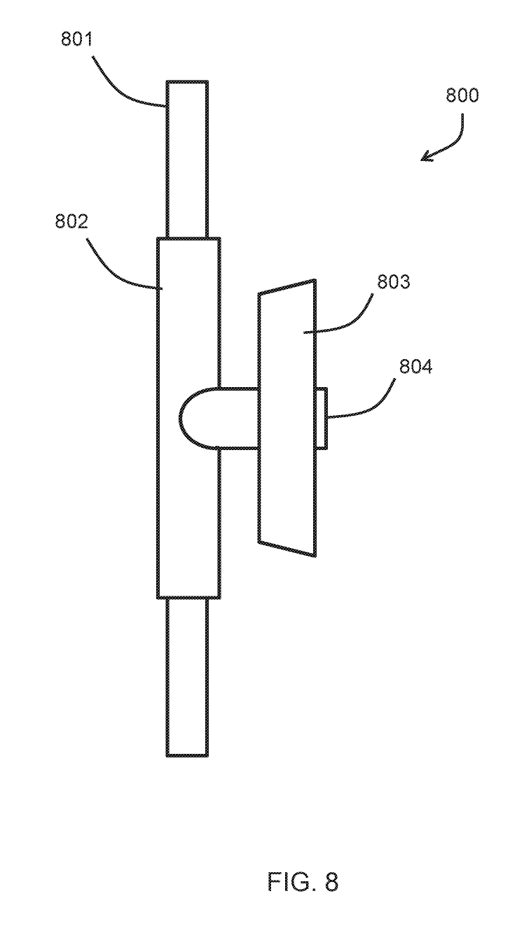

FIG. 8 illustrates a valve feedback system 800 according to another embodiment of the invention. The valve feedback system 800 includes tubing 801 and a valve device 802. The valve device 802 includes a knob 803 which is manipulated by a user to control fluid flow through the tubing 801.

In one embodiment the valve device 802 further includes a valve status indicator 804, such as a light or speaker, to emit one or both of light and sound based on the position of the knob 803.

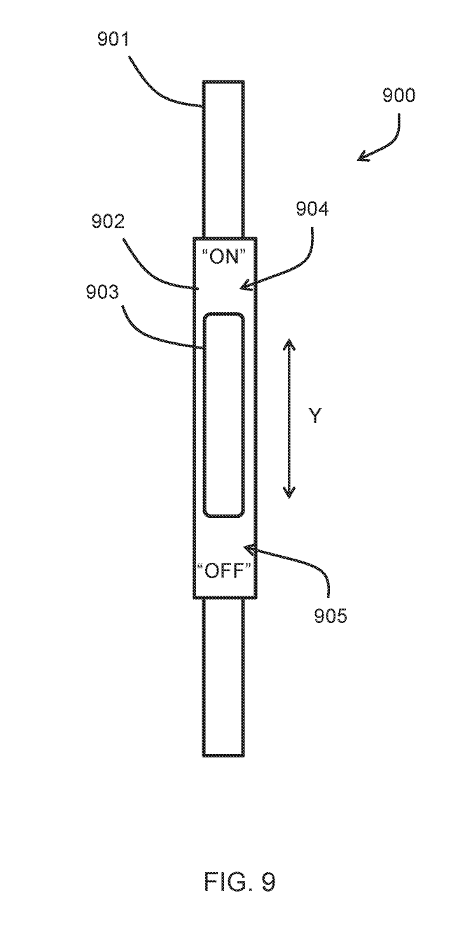

FIG. 9 illustrates a valve feedback system 900 according to another embodiment of the invention. The valve feedback system 900 includes tubing 901 and a valve device 902. The valve device includes a valve user control 903 to control fluid flow through the tubing 901. The valve user control 903 is slid along the fluid flow axis Y between a first region 904 and a second region 905. In one embodiment, the first region 904 and the second region 905 are color-coded. In another embodiment, one or more of the first region 904, the second region 905, and the valve user control 903 includes a user feedback device, such as a light, to illuminate based on a state of a fluid flow constriction device that is a part of the valve device 902 (such as the fluid flow constriction device 214 of FIG. 2). For example, when the valve user control 903 is slid toward the first region 904, the first region 904 may illuminate green, or the valve user control 903 may illuminate green. Conversely, when the valve user control 903 is slid toward the second region 905, one of the second region 905 and the valve user control 903 may illuminate red, or may remain un-illuminated.

In an embodiment in which the valve user control 903 is slid along the fluid flow axis Y, the valve user control 903 may have a protruding surface to facilitate manipulation of the valve user control 903 by a user.

In another embodiment, the valve user control 903 is not a sliding switch, but is instead a rocker switch, such that each end of the rocker switch may be pressed down to control the flow state of fluid through the tubing 901.

In other embodiments, fluid flow through tubing is controlled by rotating a user control device around a fluid flow axis, or around an axis parallel to the fluid flow axis.

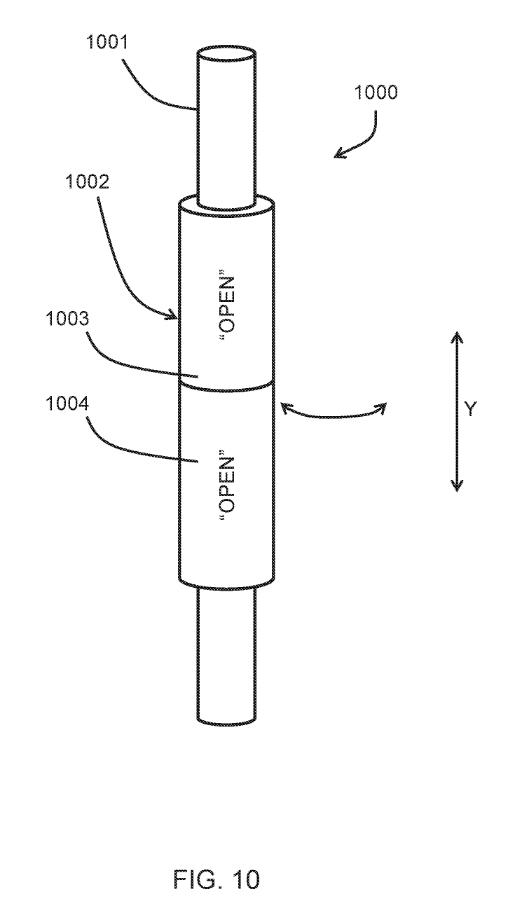

Referring to FIG. 10, a valve feedback system 1000 includes tubing 1001 and a valve device 1002. The valve device includes a first valve status indicator and user control 1003 and a second valve status indicator and user control 1004. The valve status indicator and user controls 1003 and 1004 may include both “open” regions (shown in FIG. 10) and one of the valve status indicator and user controls 1003 or 1004 may further include a “closed” region (not shown in FIG. 10, located on a back side of the device shown in FIG. 10). The valve status indicators and user controls 1003 and 1004 may be color coded according to a flow control state. For example, the “open” regions may be colored green, and the “closed” region may be colored red, black, or gray.

In one embodiment, fluid flow through the tubing 1001 is controlled by twisting the valve device 1002 such that one of the valve status indicator and user controls 1003 and 1004 rotates around the fluid flow axis Y with respect to the other. Accordingly, when both valve status indicators and user controls 1003 and 1004 have “open” regions aligned, fluid may be permitted to flow through the tubing 1001 by a fluid flow constriction device that is part of the valve device 1002, such as the fluid flow constriction device 214 of FIG. 2. In contrast, when an “open” region of one of the valve status indicators and user controls 1003 and 1004 is aligned with a “closed” region of the other of the valve status indicators and user controls 1003 and 1004, fluid is prevented from flowing through the tubing 1001.

While the words “open” are illustrated in FIG. 10 for purposes of clarity, it is understood that in some embodiments, the words may be absent, and only colors or shapes (such as arrows or triangles) may be present to represent an open or fluid-flow-permitting state of the valve device 1002.

In addition, while FIG. 10 illustrates a valve device having a cylindrical shape, embodiments of the invention encompass any shape, including a rectangular or square shape.

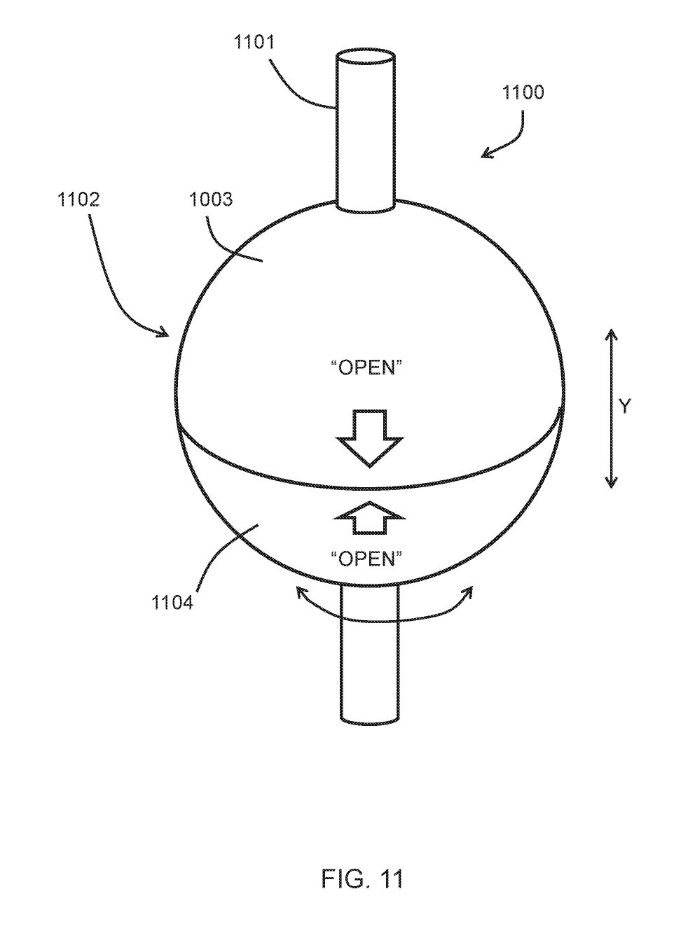

FIG. 11 illustrates a valve feedback system 1100 having a spherical shape, according to an embodiment of the invention. The valve feedback system 1100 includes tubing 1101 and a valve device 1102. The valve device 1102 includes a first valve status indicator and user control 1103 and a second valve status indicator and user control 1104. The valve status indicator and user controls 1103 and 1104 may include both “open” regions (shown in FIG. 11) and one of the valve status indicator and user controls 1103 or 1104 may further include a “closed” region (not shown in FIG. 11, located on a back side of the device shown in FIG. 11). The valve status indicators and user controls 1103 and 1104 may be color coded according to a flow control state. For example, the “open” regions may be colored green, and the “closed” region may be colored red, black, or gray.

In one embodiment, fluid flow through the tubing 1101 is controlled by twisting the valve device 1102 such that one of the valve status indicator and user controls 1103 and 1104 rotates around the fluid flow axis Y with respect to the other. Accordingly, when both valve status indicators and user controls 1103 and 1104 have “open” regions aligned, fluid may be permitted to flow through the tubing 1101 by a fluid flow constriction device that is part of the valve device 1102, such as the fluid flow constriction device 214 of FIG. 2. In contrast, when an “open” region of one of the valve status indicators and user controls 1103 and 1104 is aligned with a “closed” region of the other of the valve status indicators and user controls 1103 and 1104, fluid is prevented from flowing through the tubing 1101.

While the words “open” are illustrated in FIG. 10 for purposes of clarity, it is understood that in some embodiments, the words may be absent, and only colors or shapes (such as arrows or triangles) may be present to represent an open or fluid-flow-permitting state of the valve device 1102.

In one embodiment of the invention, an open or closed state of a valve device or valve feedback system is designed to be readily apparent from a distance, without the need for close inspection of the device. Accordingly, one or more of size, color, and shape are used to allow a user to recognize a state of the device without needing to closely inspect the device. In one embodiment, an open or closed state of the device, corresponding to a fluid-flow-permitting state and a fluid-flow-preventing state, respectively, are apparent from a distance of at least two meters by a user having 20/20 eyesight. For example, a medical practitioner applying medication to a patient may recognize a fluid-flow status of the valve device from across a room, without needing to get close to the valve device to inspect the device and determine its status.

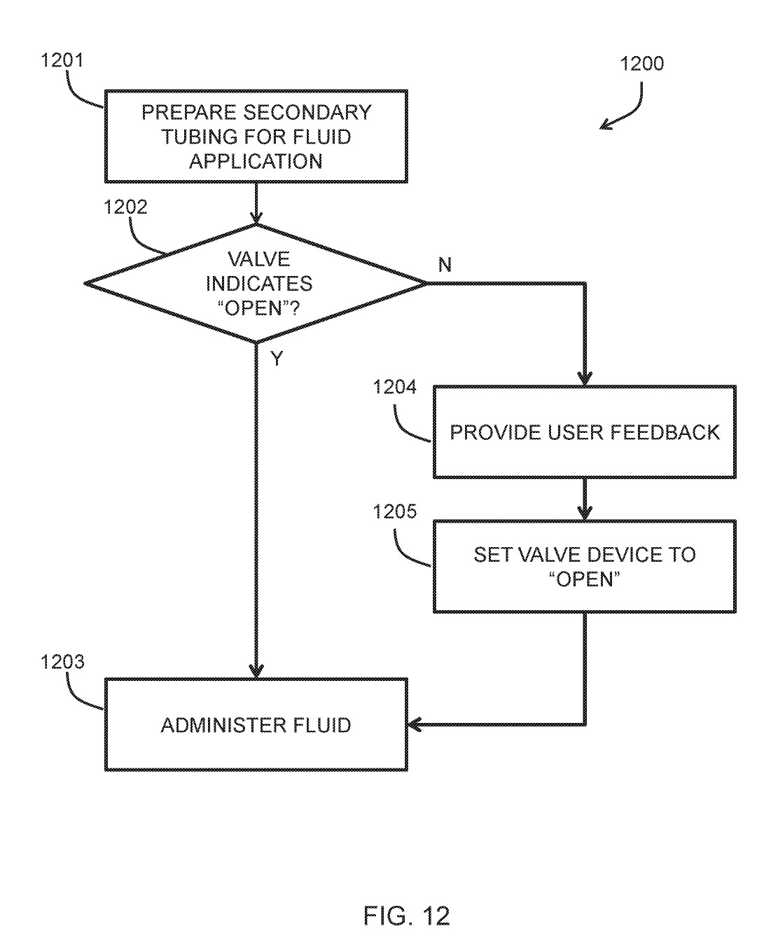

FIG. 12 illustrates a method of administering a fluid, according to an embodiment of the invention.

In operation 1201, secondary tubing is prepared for fluid application. In particular, primary tubing may be used to supply fluid from a primary fluid container to a pump, and the secondary tubing may be attached to the primary tubing to supply a fluid to the primary tubing. A valve device may be attached to the secondary tubing to prevent flow of secondary fluid from a secondary fluid container to the main tubing until such a time as a user wishes to supply the secondary fluid.

In operation 1202, it is determined whether the valve device indicates that a fluid flow constriction device is in an “open” state or a “closed” state. In operation 1203, if it is determined that the valve is “open,” fluid is administered via the valve device. In contrast, if it is determined that the valve is not “open,” feedback is provided to the user in operation 1204.

The state of the fluid flow constriction device may be determined by viewing or hearing a state of a user feedback device, which may include one or more of words, shapes, mechanical structures, lights, and sounds, that are located on or generated by the valve device.

In operation 1205, the user sets the valve device to “open,” in in operation 1203, the fluid is administered.

Embodiments of the invention relate to devices that restrict fluid flow through tubing, and it is understood that any valve may be used in embodiments of the present invention. For example, a valve may include a clamping mechanism, ball mechanism, a revolving or rotating door or tab mechanism, or any other mechanism to constrict fluid flow through a tube.

One embodiment of the present invention is directed to administering piggyback, or secondary, intravenous medications with a status display. In such a system, a main intravenous (IV) line is connected between a main fluid or medication supply container and a pump, which in turn supplies the medication or fluid at programmed rates to a patient. The secondary tubing is connected to the main tubing, so that the pump provides the combined first and second fluids or medications to the patient. A valve device located along the piggyback, or secondary, intravenous tubing is a simple on/off valve that does not include variable rate controls. Instead, it either allows fluid flow to the main IV line or prevents fluid flow to the main IV line.

When preparing the secondary fluid or medication for administration to the patient, the valve device is closed, to prevent fluid flow during preparation. However, the valve device should be opened upon preparation to allow the fluid or medication to flow via the secondary IV line to the main IV line. Accordingly, embodiments of the invention include prominent user feedback mechanisms, such as prominent colors, physical features, lights, or sounds, to indicate to a user the “open” or “closed” state of the valve device.

According to one embodiment of the invention, an intravenous fluid (IV) administration system is designed to provide user feedback to indicate a status of a fluid-flow constriction device along secondary tubing of the IV administration system. The IV administration system includes a main fluid storage container and a secondary fluid storage container. A main IV line is connected between the main fluid storage container and a fluid pump. A patient IV line is connected between the fluid pump and a patient. The secondary tubing is connected between the secondary fluid storage container and the main IV line. Fluid from the main IV line is combined with fluid from the secondary IV line before entering an input of the pump, and the combined fluid is provided to the patient.

A valve device including the fluid-flow constriction device is connected along the secondary tubing. In one embodiment, the valve device does not interrupt the secondary tubing, but instead prevents fluid flow by constricting the secondary tubing, and allows fluid flow by un-constricting the secondary tubing.

The valve device includes a user-control device and a feedback device. In some embodiments, the user-control device is integral with the feedback device, such that manipulating the user control device provides feedback to the user regarding the flow state of the fluid-flow constricting device.

In one embodiment, the fluid-flow constriction device is controllable between only an “open” state, and a “closed” state, and the user-control device does not have settings for providing fluid flow between the “open” state and the “closed” state of the fluid-flow constriction device.

In one embodiment, the user feedback device provides feedback to a user regarding an open or closed state of the fluid-flow constriction device by one or more of different colors, such as red to indicate “closed” and green to indicate “open,” different shapes, and different mechanical features. For example, in one embodiment, when the fluid-flow constriction device is closed, a prong protrudes perpendicularly from a fluid-flow axis of the secondary tubing, while when the fluid-flow constriction device is open, the prong does not protrude perpendicularly from the fluid-flow axis of the secondary tubing.

In other embodiments, the user feedback device includes one or more of electrical lights and a sound device to provide notice to a user of the open or closed state of the fluid-flow constriction device.

While one or more embodiments have been shown and described, modifications and substitutions may be made thereto without departing from the spirit and scope of the invention. Accordingly, it is to be understood that the present invention has been described by way of illustrations and not limitation.

1. A fluid administration system, comprising:
a fluid pump;
main tubing connected between a fluid storage container and an input of the fluid pump to supply main fluid to the fluid pump;
secondary tubing connected between a second fluid storage and the main tubing to supply a secondary fluid to the main fluid upstream of the fluid pump; and
a valve device located along the secondary tubing, the valve device comprising:
a fluid flow constriction device configured to allow fluid flow through the secondary tubing in an “open” position and to prevent fluid flow through the secondary tubing when in a “closed” position;
a user-control device configured to control a position of the fluid flow constriction device between the “open” position and the “closed” position; and
a user feedback device configured to display an open and closed state of the fluid flow constriction device with a color code, having a first color representing the “open” position and a second color representing the “closed” position.

2. The fluid administration system of claim 1, wherein the first and second color are located physically on the user feedback device, such that manipulation of the user-control device exposes the first color and conceals the second color based on the user-control device being in a first position, and the user control device exposes the second color and conceals the first color based on the user-control device being in a second position.

3. The fluid administration system of claim 1, wherein the user feedback device includes a light that illuminates to display at least one of the first and second colors according the first position and the second position.

4. The fluid administration system of claim 1, wherein the user feedback devices includes an audio device to emit an alarm based on the user-control device being in a position corresponding to the “closed” position of the fluid flow constriction device.

5. The fluid administration system of claim 1, wherein the user-control device includes a knob configured to be turned to a first position corresponding to the “open” position and to a second position corresponding to the “closed” position.

6. The fluid administration system of claim 1, wherein the user-control device includes a user interface structure configured to be rotated around an axis perpendicular to a fluid-flow axis to change the fluid flow constriction device between the “open” position and the “closed” position.

7. The fluid administration system of claim 1, wherein the user-control device includes a rocker switch to change the fluid flow constriction device between the “open” position and the “closed” position.

8. The fluid administration system of claim 1, wherein the user-control device includes a user interface structure configured to rotate around a fluid-flow axis to change the fluid flow constriction device between the “open” position and the “closed” position.

9. The fluid administration system of claim 1, wherein the user-control device includes a user interface structure configured to slide in a direction parallel to a fluid flow axis to change the fluid flow constriction device between the “open” position and the “closed” position.

10. The fluid administration system of claim 1, wherein the valve device is configured to be attached to the secondary tubing without breaking the secondary tubing.

11. The fluid administration system of claim 1, wherein the valve device has an input configured to be connected to a first end of the secondary tubing configured to supply the secondary fluid from the second fluid storage, and an output configured to be connected to a second end of the secondary tubing configured to supply the secondary fluid to the main tubing.

12. A valve device, comprising:
a fluid flow constriction device configured to allow fluid flow through secondary tubing in an “open” position and to prevent fluid flow through the secondary tubing when in a “closed” position;
a user-control device configured to control a position of the fluid flow constriction device between the “open” position and the “closed” position; and
a user feedback device configured to display an open and closed state of the fluid flow constriction device with a color code, having a first color representing the “open” position and a second color representing the “closed” position.

13. The valve device of claim 12, wherein the first and second color are located physically on the user feedback device, such that manipulation of the user-control device exposes the first color and conceals the second color based on the user-control device being in a first position, and the user control device exposes the second color and conceals the first color based on the user-control device being in a second position.

14. The valve device of claim 12, wherein the user feedback device includes a light that illuminates to display at least one of the first and second colors according the first position and the second position.

15. The valve device of claim 12, wherein the user feedback devices includes an audio device to emit an alarm based on the user-control device being in a position corresponding to the “closed” position of the fluid flow constriction device.

16. The valve device of claim 12, wherein the user-control device includes a knob configured to be turned to a first position corresponding to the “open” position and to a second position corresponding to the “closed” position.

17. The valve device of claim 12, wherein the user-control device includes a user interface structure configured to be rotated around an axis perpendicular to a fluid-flow axis to change the fluid flow constriction device between the “open” position and the “closed” position.

18. The valve device of claim 12, wherein the user-control device includes a user interface structure configured to rotate around a fluid-flow axis to change the fluid flow constriction device between the “open” position and the “closed” position.

19. The valve device of claim 12, wherein the valve device is configured to be attached to the secondary tubing without breaking the secondary tubing.

20. A method for applying a secondary fluid, comprising:
initiating a flow of a primary fluid through primary tubing between a primary fluid container and a fluid pump;
connecting secondary fluid tubing between a secondary fluid container and the primary tubing;
determining from a color indicator of a valve device connected along the secondary tubing whether a fluid flow through the secondary tubing is constricted; and
based on determining from the color indicator of the valve device that the fluid flow is constricted, manipulating a user-control device of the valve device to change a state of the fluid flow from constricted to un-constricted flow through the secondary tubing.
